(19) United States
(12) Patent Application Publication
(10) Pub. No.: US 2007/0243272 A1
(43) Pub. Date: Oct. 18, 2007

(54) METHOD FOR TREATING COLON CANCER WITH RICE BRAN COMPOSITION

(75) Inventors: Reddy Sastry V. Cherukuri, Folsom, CA (US); Rukmini Cheruvanky, Folsom, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: NutraCea, El Dorado Hills, CA (US)

(21) Appl. No.: 11/677,018

(22) Filed: Feb. 20, 2007

Related U.S. Application Data
(63) Continuation of application No. 11/375,411, filed on Mar. 13, 2006.

(51) Int. Cl. A61K 36/00

(52) U.S. Cl. 424/750

(57) ABSTRACT

A nutritional fiber derived from stabilized rice bran is described, which is beneficial for treating colon cancer in an individual. Methods for decreasing the likelihood of colon cancer are also described.

### CROSS-REFERENCE TO RELATED APPLICATIONS

This application is a continuation of U.S. patent application Ser. No. 11/375,411, filed Mar. 13, 2006, the teaching of which is hereby incorporated by reference in its entirety for all purposes.

### BACKGROUND OF THE INVENTION

Dietary fiber is a heterogeneous group of substances which have only one common characteristic: the non-digestibility in the small bowel. With one exception, all fibers are non-caloric carbohydrate polymers (poly- or disaccharides). Some fibers are water-soluble, others are insoluble. This property is associated with physiological effects. Soluble (viscous) fibers can bind water and thus form hydrocolloids or gels, insoluble fibers cannot form gels.

Dietary fibers also play an essential role in the physiology of the gastrointestinal tract. They modify the absorption of nutrients (particularly carbohydrates and lipids) in the small bowel. They accelerate the gut transit time and determine stool composition and quantity. They are the main nutritional source for the colonic microflora. During the bacterial fermentation short-chain fatty acids are formed which are essential for nutrition and integrity of the colonocytes and for colonic function. Moreover gases, detoxifying enzymes, antioxidants and carcinogen-inactivating compounds arise. The most important fibers are cellulose, hemicellulose, pectin, guar, psyllium, beta-glucan, lignin and digestion-resistant starch; they are present in varying amounts in plant foods and in fiber preparations. The usual daily intake of dietary fiber in Europe and the United States amounts to only 15-20 grams, while health authorities and nutrition societies recommend a reference value of at least 30 grams per day. Dietary fibers are applied as food-integrated substances, as supplements to be taken with food, and as purified substances.

The health benefits of dietary fiber are associated with a decreased incidence of certain diseases such as cardiovascular disease, diabetes, cancer of several organs, kidney stones as well as an improvement in colon health. Although certain dietary fiber formulations are available, a need exists for formulations with a practical shelf-life and which maximizes the benefits of fiber to consumers of such formulations.

### SUMMARY OF THE INVENTION

The present invention provides a stabilized rice bran fiber and methods of treating or preventing various maladies through the consumption or administration of the fiber.

In one embodiment, the invention provides a rice bran fiber composition derived from pre-digested rice bran, wherein said composition comprises soluble rice bran fiber, insoluble rice bran fiber, and fructo-oligosaccharides; and wherein said composition is substantially lactose-free and substantially gluten-free. In a related embodiment, the composition of the invention comprises at least 35% rice bran fiber, on a weight percentage basis (i.e., “w/w”). In another related embodiment, the composition of the invention comprises between 35% and 60% w/w rice bran fiber. In yet another related embodiment, the composition of the invention comprises between 35% rice bran fiber and 50% rice bran fiber. In still another related embodiment, the composition of the invention comprises between 35% rice bran fiber and 45% rice bran fiber. In yet another related embodiment, the composition of the invention comprises omega-3 and omega-6 fatty acids. Each of the aforementioned embodiments of the composition of the invention may further comprise phytonutrients and antioxidants.

In one embodiment of the rice bran composition of the invention, the composition comprises approximately 42% insoluble rice bran fiber, 1.0% soluble rice bran fiber, 21% protein, 13% fat, and 1% available carbohydrates. The invention also provides a composition which comprises the rice bran fiber of Table 5.

The invention provides a method of reducing the likelihood of cancer in a subject, comprising administering one of the aforementioned rice bran compositions of the invention to a subject. For example, in one embodiment, the invention provides a method of reducing the likelihood of cancer in a subject comprising administering a rice bran fiber composition to a subject, wherein the composition comprises soluble rice bran fiber, insoluble rice bran fiber, and fructo-oligosaccharides; and wherein the composition is substantially lactose-free and substantially gluten-free; and wherein 35-60% of the composition consists of soluble and insoluble rice bran fiber by weight. In one embodiment of the method, the subject has a higher risk of cancer than an average or normal subject. In a related embodiment, the cancer is selected from the group consisting of cancer of the breast, prostate, lung, colon, rectum, bladder, pancreas, liver, kidney, skin, mouth, and non-Hodgkin lymphoma. In one embodiment of the method, the cancer is colon cancer. In a related embodiment, approximately 10 to approximately 50 grams of the composition are administered to a subject on a daily basis. In yet another related embodiment, the higher risk of cancer in the subject is determined prior to the administration of the rice bran composition. In yet another related embodiment, the determination comprises the use of a genetic test. In yet another related embodiment, the administration of the composition facilitates the apoptosis of cancer cells in the colon.

In one embodiment, the invention also provides a method of improving gastro-intestinal and colon health in a subject, comprising administering one of the aforementioned rice bran compositions to a subject. In another embodiment, the invention provides a method of facilitating the growth of bifido bacteria in the intestines, comprising administering a rice bran composition of the invention to a subject. In a related embodiment, the fermentation of the rice bran fiber in the intestines of the subject lowers the pH in the subject's intestines. In another embodiment, the administration of the rice bran fiber leads to an increase in the measured intestinal enzyme activity in the subject. In yet another embodiment, the administration of the rice bran fiber composition of the invention improves the absorption of sodium and water in the intestines.

In another embodiment, the invention provides a method of decreasing the likelihood of gall stone and kidney stone formation in a subject, comprising administering one of the aforementioned rice bran compositions of the invention to a subject. In yet another embodiment, the administration of said rice bran fiber induces the synthesis of B12-vitamins in said subject.

### BRIEF DESCRIPTION OF THE FIGURES

FIG. 1 illustrates the key differences between flattened, injured or immature absorptive intestinal cells versus tall, healthy, mature absorptive intestinal cells.

FIG. 2 illustrates the cancer blocking and/or suppressive effects of phytochemicals present in the stabilized rice bran fiber compositions described herein. Specifically, the illustration shows how phytochemicals act at different stages of carcinogenesis.

FIG. 3 illustrates the steps in the standard Ames assay.

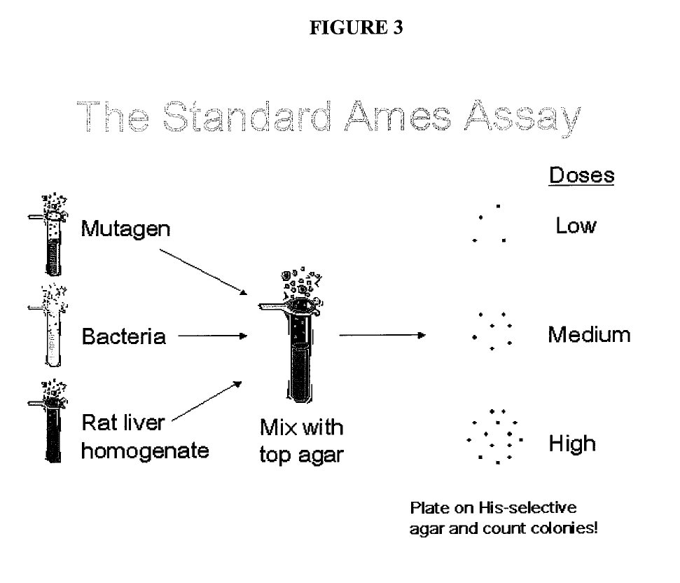

### DESCRIPTION

I. Definitions

As used herein the term “stabilized rice bran derivative insolubilized fraction” refers to a fraction of stabilized rice bran produced during a partitioning process. Specifically, after the stabilized rice bran aqueous slurry is enzymatically treated as discussed fully below, it is then pumped into a centrifuge where the insoluble fraction precipitates out of the aqueous solution. The insoluble fraction is collected and then dried, and subsequently ground into a powder. This powder is the insoluble portion. In a preferred embodiment, the constituent parts and their percentages are listed in Table 5. The process for isolating this fraction from rice bran is described in Example 1.

As used herein the term “stabilized rice bran derivative solubilized fraction” refers to a fraction during a partitioning process. Specifically, after the stabilized rice bran aqueous slurry is enzymatically treated, it is then pumped into a centrifuge where the insoluble fraction precipitates out of the aqueous solution. The aqueous material is pumped to a dryer and then dried. This dried aqueous portion produces the soluble fraction.

As used herein the term “triglyceride” means a lipid or neutral fat consisting of glycerol combined with three fatty acid molecules.

The rice bran fiber of the present invention is a combination of both soluble and insoluble fiber. It has significant amount of fructo-oligosaccharides, the food for bifidobacteria to proliferate in the intestines. Rice bran fiber is associated with potent antioxidants, quality fat and hypoallergenic protein, but is free of lactose and gluten. It is an all-natural derivative of stabilized rice bran (SRB) and is nutritionally superior to any other fiber existing today (see, e.g., Table 4). In one embodiment, the invention provides a rice bran fiber known as RiceMucil™. RiceMucil™ is prepared from SRB following a non-chemical process by which the nutritionally potent fiber is separated from the soluble components into a fine feathery powder (U.S. Pat. No. 6,350,473, incorporated by reference herein, in its entirety for all purposes). A detailed description of this embodiment of rice bran fiber is provided in Table 5.

In another embodiment, the stabilized rice bran fiber prepared according to the above-described process comprises the following ingredients per 10 gram serving: 4.2 grams insoluble fiber; 0.1 grams soluble fiber; 2.1 grams protein; 1.3 grams fat; and 1.0 grams available carbohydrates. The relative amounts of the ingredients are approximate and the amount of each can be changed by approximately 10% without substantially altering the beneficial properties of the fiber.

II. Detailed Description

The physiological attributes of dietary fiber depend on their molecular design and solubility in water. When these undigested dietary fibers reach the colon, the bacteria ferment the insoluble fibers, producing short chain fatty acids (i.e., acetic, propionic and butyric acids) and reduce the pH to acidic levels. These acids known as volatile fatty acids are used as an energy source by the mucosal cells of the colonic epithelium. The high viscosity in the gut created by the soluble fiber results in a decreased uptake of glucose and is beneficial to the patient with diabetes. The absorption of lipids through the same mechanism lowers cholesterol.

Dietary fiber adds to the bulk of the diet by assisting in the transit of food into the gastrointestinal tract. It retains water and softens the stool for easy excretion (Normand, F. L. et al., Food Technology 2:90-99 (1987)). Dietary fiber plays an important role in weight reduction as it expands in the stomach occupying space thereby increasing satiety and decreasing appetite. Dietary fiber contains no calories as it is not digested in the human gut. Dietary fiber helps in healthy weight loss as it creates satiety and decrease in appetite.

Two types of fibers are essential for their functional role in the diet. A combination of soluble and insoluble fibers is required to achieve maximum health benefits. Suggested practices for maintaining a healthy intake of dietary fiber include (1) taking a supplement with a balanced matrix of soluble and insoluble fiber; (2) ingesting 30-35 grams/day total dietary fiber (TDF) which includes 8-10 grams of soluble fiber; and (3) drinking 8 glasses of water a day since fiber supplements absorb water. If an adequate amount of water is taken, the fiber will utilize intestinal fluids resulting in electrolyte imbalance.

Insoluble fiber consists of celluloses, hemicelluloses and lignins. Lignins are mature cell wall matrices consisting of condensed polyphenols. Whole grains, rice bran, and wheat bran are rich in insoluble fiber. Mature vegetables, beans, apples and root vegetables are other sources of insoluble fiber. Insoluble fiber passes through the gut unchanged and reaches the colon. Insoluble fiber also helps in maintaining gastrointestinal and colon health (Folino, M. et al., J. Nutrition, 125(6):1521-8 (1995)). It helps in the fermentation of undigested food in the colon, binds with the bile salts and bile pigments for excretion, and aids in the proliferation of bifido bacteria by providing an acidic pH.

During the anaerobic bacterial fermentation of undigested food in the colon, insoluble fiber produces short chain fatty acids (SCFAs) like propionic acid and butyric acid (unlike soluble fibers which produce gases such as carbon dioxide and methane), acidifying the lower luminal pH. This acidic environment in the colon has several beneficial effects such as promoting the growth of friendly bacteria, stimulating cell turn over between the villi and increasing the production of RNA, DNA and protein by the cells. SCFAs also stimulate pancreatic enzyme secretion, increase sodium and water absorption in the colon, and induce the synthesis of B-complex vitamins, especially B12. Additionally, SCFAs in the colon induce the synthesis of natural killer cells (NF-kB) which are involved in the natural death of cancer cells. This property helps in preventing colon and prostate cancer by gene regulation and apoptosis (cell death of the cancer cells). Insoluble fiber prevents diverticulitis, colon/rectal polyps (Harvard study with 43,881 US male health professionals ranging from 40-75 years), IBS (inflammatory bowel syndrome), ulcerative colitis and cancer. A condition consisting of inflamed colon may arise from food allergies, stress or bacterial/viral infections.

Soluble fiber is soluble in water, forming a highly viscous gel-like fluid with food in the small intestines. The high viscosity is responsible for the delayed absorption of glucose in the intestines and reduces the post prandial glucose levels in the diabetics. This high viscosity also helps in the reduction of the serum cholesterol and triglyceride levels and increasing HDL levels (Madar, Z., Am. J. Clin. Nutr., 38:388 (1983)). Fibers, both soluble and insoluble, prevent the re-absorption of the bile acids from the small intestines back into circulation thereby reducing the circulating cholesterol. Fibers also dissolve the gallstones in the gallbladder.

Examples of soluble fibers are gums, pectins, mucillages, galactomannans, arabinogalactans, beta glucans, from barley and non-starchy water-soluble polysaccharides containing hemicelluloses. Most fruits, vegetables, barley, psyllium and oat bran have soluble fibers. Soluble fiber such as pectin is composed of repeated units of galactouronic acids that have been methylated and have β 1-4 covalent linkages. Gums and mucillages are also soluble fibers composed of hexose and pentose units with β 1-4 covalent linkages.

In addition to 42% fiber, the RiceMucil™ embodiment of the present invention contains proteins, antioxidants and phytonutrients, such as gamma oryzanol, inositol hexaphosphate (IP6), and phytosterols, which provide excellent nutrition to the gut and help to maintain a healthy and clean gut. It also contains a short chain sugar complex, known as fructo-oligosaccaride (FOS), which is not digestible in the gut and serves as food to the friendly bacteria, such as Lactobacillus acidophilus or Lactobacillus sporogens.

These friendly bacteria aid in the fermentation of the undigested food around the intestinal villi, producing short chain fatty acids, such as butyric and propionic acids in the gut. Scientific data indicates that the short chain fatty acids play an important role in the prevention of colon cancer. Other commercially available dietary fibers produce carbon dioxide and methane gases during colonic fermentation, resulting in abdominal distention and discomfort.

RiceMucil™ in the diet prevents constipation and irregular bowel movement. It provides bulk to the waste and decreases the colonic transit time by several-fold, thereby reducing the risk of colorectal cancer.

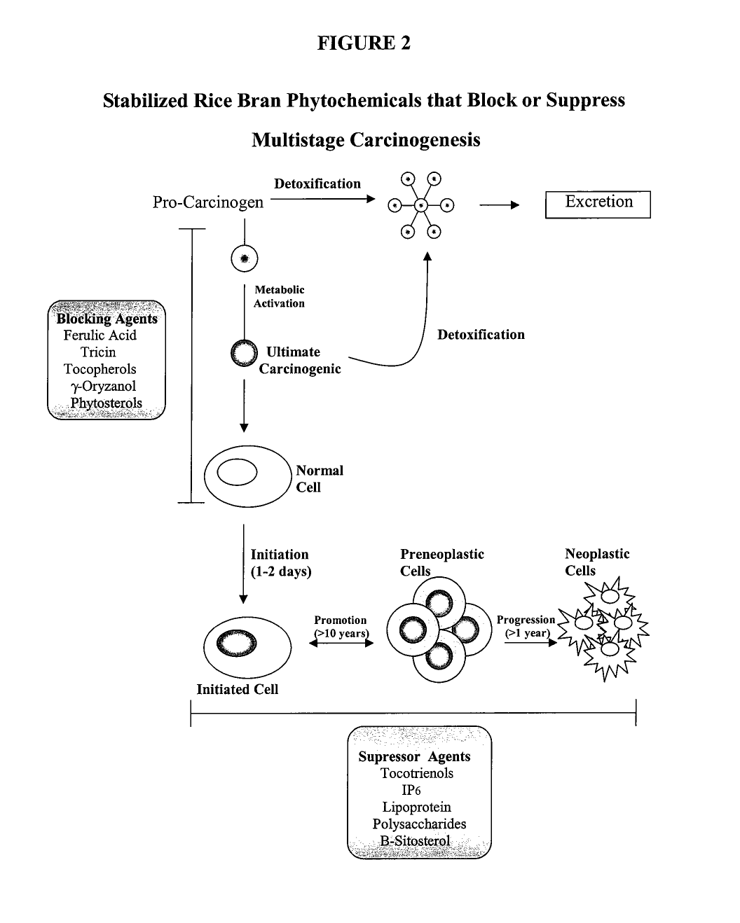

Cancer is a result of free radical damage to DNA and protein in various organs of the body. Cancer formation occurs in three stages: (1) an initiation stage, (2) a latent stage of progression and (3) tumor formation stage. Certain natural products are known to inhibit cancer by two major mechanisms. Specifically, the products may act as blocking agents and/or as suppressing agents (see FIG. 2). By blocking attacks on DNA and protein by active carcinogens, cancer can be prevented at the initiation stage itself. Even after the formation of DNA adducts, cancer can be arrested during the latent period by the suppressor mechanism, preventing the proliferation of diseased cells.

Components of the rice bran fiber compositions described herein are involved in the above-mentioned blocking and suppressing mechanisms. For instance, ferulic acid increases liver microsomal phase 2 detoxifying enzymes several fold and inhibits phase 1 carcinogen activating enzymes (Manorama, 1993). In the presence of sufficient amounts of ferulic acid and similar compounds, a carcinogen entering the body cannot reach the DNA because it is not activated to a reactive metabolite. The body's detoxification mechanisms can eliminate the carcinogen before it is capable of doing harm. Thus, this blocking mechanism prevents cancer formation.

In contrast, as described above, the suppressor mechanism acts on previously formed DNA adducts to suppress the progression of cancer. For example, the signal transduction pathway created by T3, IP6 and other phytonutrients, appears to switch the cell programming from a proliferative to a differentiation pathway, which can reverse cancer progression. IP6 has been shown to act both as a blocking agent and as a suppressing agent. Data on breast cancer, lung cancer, and colon cancer, as well as the cancer of several organs (Shamsuddin, 1995), shows a strong chemopreventive effect of IP6 treatment, where IP6 appears to function both as a blocking agent and as a suppressive agent.

Rice bran polyphenols also have a chemopreventive effect (Hudson et al., 2000). Tocotrienols are another group of rice bran phytochemicals having a chemopreventive effect which have been shown to inhibit breast cancer (Nesaratnam et al., 1997). The polysaccharides of rice bran include alpha glucan, the anti-tumor effect of which was demonstrated by the inhibition of gastrointestinal carcinogenesis (Akeshita, 1992). Rice bran agglutinin was shown to induce apoptosis of cancer cells by the mechanism of cell cycle dysregulation (Myoshi et al., 2001). In addition to these compounds, rice bran includes potent antioxidants that prevent the free radical attack at several stages of carcinogenesis. Some of these phytochemicals (e.g., phytosterols) get incorporated into cell membranes and improve the cell integrity and fluidity of the cell membrane. This prevents the carcinogens from entering the cell. An anti-tumor polysaccharide from rice bran has been isolated (Ito et al, 1985) and has been demonstrated to have anticancer and immune enhancing properties (Nakumura, 1992). Another anti-tumor lipoprotein fraction of rice bran has been shown to induce apoptosis and inhibit the growth of cultured human endometrial adenocarcinoma cells (Fan et al., 2000).

In addition to their cancer preventative effects, the rice bran fiber compositions of the present invention are useful for lowering cholesterol and treating hyperlipidemia. RiceMucil™ was tested for RiceMucil™ its cholesterol lowering effect in an eight-week human study. A total of 20 individuals with clinically established Type 1 diabetes, and 26 individuals with Type-2 diabetes, with lipid abnormalities, were treated with RiceMucil™ by administering in two equally divided doses of 10 grams each, one taken before breakfast and another before dinner in milk/juice for a period of eight weeks. RiceMucil™ produced significant reduction in total cholesterol, LDL cholesterol, apo B, and triglycerides in both Type 1 and Type 2 diabetics with percentage reductions ranging between 7-18%. There was no change in HDL cholesterol. Results of the above study indicate clearly that RiceMucil™ controls hyperlipidemia very effectively (see Cheruvanky et al., U.S. Pat. No. 6,126,943).

RiceMucil™ and other embodiments of the rice bran fiber of the present invention reduces blood cholesterol by premature emptying of the gall bladder and trapping the bile salts, which thereby prevent re-absorption. It is believed that the high quality fiber in the compositions disclosed herein, along with the major bioactive compounds (such as tocopherols, tocotrienols, gamma oryzanol, phytosterols, and inositol) present in the product, synergistically help in the management of heart diseases.

A diet rich in fiber also helps to prevent gallstone formation. This is due to improved cholesterol levels, which prevent high cholesterol deposits (in crystallized form) from lodging in the gall bladder. Researchers have shown that the effectiveness of a high rice bran dietary fiber intake in reducing renal calcium excretion (Jahnen, A. et al., Urol. Res. 20:3-6 (1992)). A highly nutritious dietary fiber complex derived from rice bran, such as RiceMucil™, helps in preventing gall stone formation by keeping the cholesterol levels under check, as well as by effectively reducing renal calcium excretion, which are the two main causes of gallstones.

RiceMucil™ helps to stabilize blood sugar levels in Type 1 and Type 2 diabetics because it contains the right type of fiber, which can slow the rate of sugar absorption into the blood stream, thereby reducing the level of insulin required to process the food at any given moment.

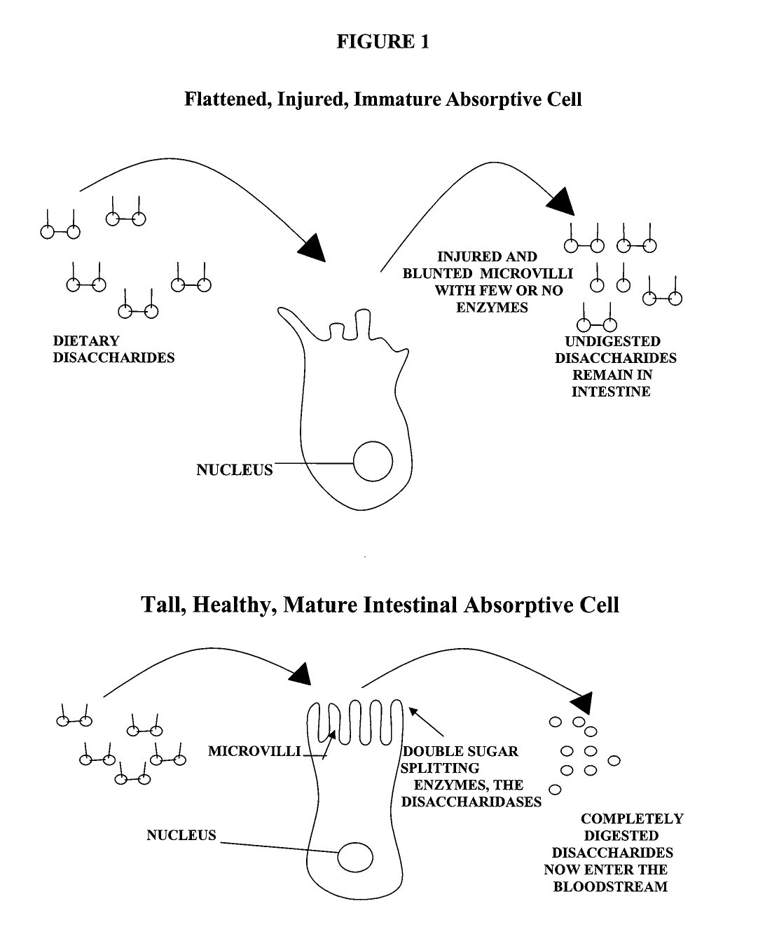

In another embodiment, the stabilized rice bran fiber compositions of the present invention stimulate microvilli in the intestine and extend their age (FIG. 1). Other fibrous compositions and diets can erode microvilli and inhibit the ability of microvilli to synthesize and maintain active disaccharidases, either directly or by facilitating the invasion of enteroadherent bacterial pathogens (see, e.g., Fleckenstein and Kopecko, J Clin Invest, 107:27-30). The presence of eroded or damaged microvilli thus impairs the body's ability to digest disaccharides.

Constipation and irregularity of bowel movements are also major consequences of fiber-deficient diets. Ingesting the correct amounts and types of fibers increases the fecal bulk while also absorbing water into the large intestine. This creates a softer stool that exerts less pressure on the colon walls and is more easily excreted. RiceMucil™ contains 42% insoluble fiber, and is an ideal help for constipation. It increases fecal bulk and reduces the length of time of gastric emptying, thereby giving instant relief to people suffering from constipation.

Fiber is preferably taken between meals or on an empty stomach in the morning and evening. Fiber contains phytates and these tend to absorb minerals such as calcium, magnesium, manganese, and iron. Consumption of fiber is therefore most effective when taken on an empty stomach.

Twenty grams of RiceMucil™ a day, when given in two equal doses of 10 grams each before breakfast and before dinner to Type 1 and Type 2 diabetic patients for eight weeks, significantly reduced glucose parameters (Cheruvanky et al., U.S. Pat. No. 6,303,586 B1).

RiceMucil™ taken half an hour before mealtime or with meals, forms the bulk of food replacing high-calorie diets. A high fiber, low fat, low calorie meal is recommended for weight control. RiceMucil™, which has 20% protein and significant quantities of gamma oryzanol and several other phytonutrients, is an effective diet for weight management.

RiceMucil™ is all natural, non-genetically engineered, chemical-free, and manufactured according to the strict GMP conditions of FDA. Every batch is tested for quality and safety in the laboratory.

Because of the antimutagenic properties of the rice bran-derived composition of the invention, physicians who have diagnosed a patient as having a form of cancer, e.g., colon cancer, may recommend that the patient be treated with (e.g., consume) one or more of the rice bran-derived compositions of the present invention. A physician may also recommend that patients who are at higher risk of a cancer, e.g., colon cancer, also be treated with (e.g., consume) one or more of the rice-bran derived compositions of the present invention. A patient with a higher risk of cancer is a patient who is more likely than an average member of the population to develop any particular form of cancer, i.e., the risk could be the risk of a future diagnosis of a specific cancer such as colon cancer or lung cancer, or the risk of a future diagnosis of cancer generally. Patients at higher risk of cancer include, without limitation, patients who smoke, patients who are genetically more susceptible to cancer, patients who work in environments where they are exposed to a higher concentration of mutagenic substances than patients in the general population, etc.

In summary, the present invention provides rice bran fiber compositions which are hypoallergenic, lactose-free and gluten-free, as well as methods of administering those compositions for the purpose of achieving bowel regularity and relieving constipation; normalizing blood glucose levels; normalizing serum cholesterol and lipid levels; reducing the risk of coronary heart disease; achieving healthy weight loss; preventing cancer of the colon; improving gastrointestinal and colon health; improving the colonization of probiotics (friendly bacterial colonies) in the intestines due to a significant amount of fructooligosaccharides (FOS); improving sodium and water absorption in the colon; improving the intestinal enzyme production due to the low pH created by short chain fatty acids (SFA); preventing gallstone and kidney stone formation; and inducing the synthesis of B-Complex vitamins.

### EXAMPLE 1

### Preparation of Rice Bran Derivatives

In order to generate the rice bran derivatives for use in the present invention, the rice bran is first stabilized, and then it is further separated into at least two fractions. These include, but are not limited to, a stabilized rice bran soluble derivative and a stabilized rice bran insoluble derivative. Preferably, the separation into the rice bran derivatives includes a nonchemical process i.e., an enzymatic process. In this process, partitioning or fractionation preferably proceeds as outlined hereinafter.

The stabilized rice bran is made into about a 15% to about 35% slurry, preferably, a 20-25% slurry with potable water. An enzyme, which can include, but is not limited to, a dextranase, a maltase, a .alpha.-amylase, and various other carbohydrate cleaving enzymes, is added to the batch converting the starch to dextrins. The slurry is heated to about 150° F. to about 200° F. using, for instance, a steam injection cooker, a heat exchanger, or other heating method. The slurry is then pumped to a horizontal centrifuge wherein the insoluble fraction is separated. The insoluble fraction is collected and then dried on a belt dryer, and subsequently ground into a powder. This powder is the stabilized rice bran insoluble fraction. The aqueous material is pumped to a drum dryer and then dried. This dried aqueous portion produces the stabilized rice bran solubilized fraction.

The enzyme treated stabilized rice bran can be generated using the rice bran slurry as described above. As such, in another aspect, the present invention relates to the process for making an enzyme treated stabilized rice bran derivative, comprising: admixing stabilized rice bran with an aqueous solution to form about a 15% to about a 35% aqueous rice bran slurry, preferably a 20% to about a 30% aqueous rice bran slurry w/w; adding an enzyme to the aqueous rice bran slurry to convert starch to dextrin, thereby forming an enzyme treated slurry and then directly drying the enzyme treated slurry to form an enzyme treated stabilized rice bran derivative.

In a preferred embodiment of the foregoing process, after the enzyme is added to the slurry, the slurry is heated to about 100° F. to about 200° F. Preferably, the slurry is heated to about 150° F. to about 200° F. The slurry is then dried, wherein the drying is accomplished by a process such as belt drying, spray drying, drum drying and air drying. The drum drying process is preferred.

These stabilized rice bran derivatives are also available commercially from the NutraCea company of El Dorado Hills, Calif. Specifically, the insoluble derivative of stabilized rice bran is available as RiceMucil™ Fiber Complex and the soluble derivative is available as RiSolubles®.

The stabilized rice bran derivatives can take a variety of forms. They can be a powder, a food, a food supplement, a medical food, a liquid, a beverage, an emulsion or mixture thereof. In addition, they can be incorporated into other edible materials. To incorporate the rice bran derivative into the diet of a mammal various options include, but are not limited to, simply sprinkling the derivative on another food substance (i.e., salad, bread, cereal, etc.) being a major ingredient in a multigrain ready to eat cereal, incorporating it into a baked product (breads, muffins, waffles, etc), pasta, healthy dessert and snacks (athletic bar, healthy drink, etc.) and high fiber foods.

Stabilized rice bran contains about 18-23% fat, about 23-35% dietary fiber, about 12-16% protein, about 8-36% total carbohydrate and many potent microcomponents. Rice bran solubles contains about 15-40% fat, preferably 23-30% fat; about 0% to 25% dietary fiber, preferably about 0-20% dietary fiber; about 0% to 15% protein, preferably 6-9% protein and 25% to about 80% carbohydrates, preferably about 27-66% simple carbohydrate and is a water soluble fraction. Stabilized rice bran insoluble derivative contains about 5%-20% fat, preferably 11-16% fat; about 40-65% dietary fiber, preferably 40-60% dietary fiber, and about 10-30% protein, preferably 18-22% protein (see Table 5).

### EXAMPLE II

### Antimutagenic Effects of RiceMucil™

This Example describes tests used to demonstrate the antimutagenic properties of RiceMucil™ and its components.

A. Overview of Salmonella mutagenicity test. The Salmonella mutagenicity test, or bacterial reverse mutation assay, is also commonly known as the Ames test (after Dr. Bruce Ames, who developed the test with is colleagues in UC Berkeley in the 1970s). The principle of the test is to expose histidine-dependent Salmonella typhimurium strains (the tester strains, which have artificially induced point mutations) to a compound to be examined in a histidine (His) deficient medium. His-independent bacterial colonies may arise from spontaneous reversions (backward mutations) or chemically induced reversions.

The Ames test is a screening assay for carcinogens that uses bacteria to detect chemical mutagens. It is based on the premise that most carcinogens induce cancer because they are mutagens. If these agents are shown to be mutagenic for bacteria, they may also alter DNA in eukaryotic cells. In the Ames test, a strain of Salmonella typhimurium auxotrophic for histidine (his-), defective in dark repair of mutations (uvrB), and unable to synthesize a portion of the cell wall (rfa) is exposed to chemicals. The rate of reversion (back mutation) to prototrophy caused by the chemical is measured.

The test is performed by spreading a suspension of the Salmonella in soft agar over a layer of minimal medium. The suspected mutagen may be added to the soft agar along with the Salmonella or it may be placed on a disk in the center of the seeded layer after it is spread. The soft agar contains an amount of histidine that supports only a few rounds of bacterial cell division. This is essential because many mutagens work only on replicating DNA. If a back mutation to prototrophy occurs, visible colonies develop. The reversion rate is compared to a control plate (no chemicals added) and is proportional to the mutagenicity of the chemical. Many chemicals that are non-mutagenic by the Ames test can induce tumors. Some of these chemicals are metabolized to a mutagenic form in the liver. To mimic the activation process, the Ames test usually contains an extract of mammalian liver enzymes.

In a standard Ames assay a rat liver homogenate is used to provide the cytochromes P450 necessary for activation of mutagens to their reactive forms. The suspected mutagen is mixed with the homogenate, where it is metabolized to its reactive form, and then an appropriate tester strain of Salmonella typhimurium is added to the mix. The activated mutagen enters the bacterium and reacts with DNA to cause mutations; mutations in the operon for histidine biosynthesis that restore function (“reversions”) allow the bacterium to grow on histidine-deficient medium, and result in visible colonies on the assay plate. The greater the number of colonies, the more mutagenic the chemical.

The following bacterial strains are used in this assay: TA98 (frame-shift mutation in histidine gene); TA100 (base-pair substitution in histidine gene); TA1535 (base-pair substitution in histidine gene); TA1537 (frame-shift mutation in histidine gene); and TA102 (base-pair substitution in histidine gene). The two standard testing strains (TA1535 and TA1537) are used in combination with the strains, TA98, TA100 and TA102, which include the plasmid pKM101, as well as their particular histidine gene mutation. The plasmid-carrying derivatives (TA98, TA100 and TA102) have an increased sensitivity to certain mutagens because pKM101 codes for an error-prone DNA repair system. Strain TA102 has its histidine gene mutation located on a multi-copy plasmid, pAQ1.

Many chemicals are not mutagenic (or carcinogenic) in their native forms, but they are converted into mutagenic substances by metabolism in the liver. Since the Salmonella bacterium does not have the same metabolic capabilities as mammals, some test protocols utilize extracts of rat or hamster liver enzymes (S9) to promote metabolic conversion of the test chemical. This permits the investigator to determine if a chemical must be metabolized to express mutagenic activity. Some mutagenic chemicals are active with and without metabolism, while others are active only under one condition or the other.

In the Salmonella assay, a test tube containing a suspension of one strain of Salmonella typhimurium plus S9 mix or plain buffer without S9, is incubated for 20 minutes at 37° C. with the test chemical. Control cultures, with all the same ingredients except the test chemical, are also incubated. In addition, positive control cultures are also prepared; these contain the particular bacterial tester strain under investigation, the various culture ingredients, and a known potent mutagen. After 20 minutes, agar is added to the cultures and the contents of the tubes are thoroughly mixed and poured onto the surface of petri dishes containing standard bacterial culture medium. The plates are incubated, and bacterial colonies that do not require an excess of supplemental histidine appear and grow. These colonies are comprised of Salmonella that have undergone reverse mutation to restore function of the histidine-manufacturing gene. The number of colonies is counted after 2 days.

B. Mutagenicity testing of rice bran oil unsaponifiable fraction. We have carried out three experiments with rice bran oil and its unsaponifiables using a Salmonella typhimurium bacterial assay system.

Rice bran oil unsaponifiable extracts were prepared and separated into polar and non-polar fractions according to Taylor et al. (Journal of the American Oil Chemists Society, (1983) 60:576). Rice bran has 20% fat. When the fat is extracted from rice bran or rice bran fiber, the fat is known as rice bran oil. Rice bran oil concentrates all the fat-soluble phytonutrients and antioxidants of rice bran into the oil. The non-glyceride portion of the oil is known as the unsaponifiable fraction, containing concentrated rice bran phytonutrients and antioxidants of the rice bran. This unsaponofiable fraction is further separated into polar and non-polar fractions, per the protocols described in Taylor et al. (1983).

Assays were conducted using Salmonella typhimurium strains TA 98 and TA 100, plated at a density of 6.6×10⁹ bacteria per plate. The rat liver microsomal S9 extracts were prepared according to Maron and Ames (Mutat Res., (1983) 113:173-215). Reported values are the mean of four plates. The mutagens benzo(a)pyrene (5 μg/plate) and sodium azide (1 ag/plate) were used as positive controls for TA 98 and TA 100 respectively. DMSO extracts of the rice bran oil fractions (0.2 ml) was mixed with 0.5 ml of S9 mixture and 0.1 ml of overnight grown bacterial culture. The mixture was then poured on minimal, glucose agar medium plates and incubated in the dark at 37° C. After 48 hours, the number of colonies of histidine prototrophs on the plates were counted.

C. Results.

TABLE 1
Mutagenicity test in Salmonella typhimurium TA 98 strain

| Without S9 | | With S9 | |
|---|---|---|---|
| Rice bran oil | Peanut oil | Rice bran oil | Peanut oil |

| polar fxn | non-polar fxn | polar fxn | non-polar fxn | polar fxn | non-polar fxn | polar fxn | non-polar fxn |
|---|---|---|---|---|---|---|---|
| 20 ± 6 | 16 ± 2 | 26 ± 5 | 35 ± 4 | 26 ± 2 | 29 ± 3 | 30 ± 5 | 24 ± 6 |

Each of the above values represents mean number of revertants from 4 plates with their SD. No significant difference was observed between the rice bran unsaponifiables and peanut oil unsaponifiables. The TA 98 control without benzo(a)pyrene yielded 24±4 colonies; the TA 98 control with benzo(a)pyrene yielded 58±2 colonies. These data show that the constituents of rice bran unsaponifiables are non-mutagenic.

TABLE 2
Mutagenicity test in Salmonella typhimurium TA 100 strain

| Without S9 | | With S9 | |
|---|---|---|---|
| Rice bran oil | Peanut oil | Rice bran oil | Peanut oil |

| polar fxn | non-polar fxn | polar fxn | non-polar fxn | polar fxn | non-polar fxn | polar fxn | non-polar fxn |
|---|---|---|---|---|---|---|---|
| 153 ± 8 | 151 ± 88 | 136 ± 7 | 140 ± 2 | 163 ± 18 | 166 ± 13 | 152 ± 15 | 127 ± 16 |

Each of the above values represents mean number of revertants from 4 plates with their SD. No significant difference was observed between the rice bran unsaponifiables and peanut oil unsaponifiables. The TA 100 control without azide yielded 158±14 colonies; the TA 100 strain treated with azide yielded 485±18 colonies. These data show that the constituents of rice bran unsaponifiables are non-mutagenic.

TABLE 3
Mutagenicity of Rice Bran Oil before and after 6 hours heating at 180° C.

| | |
|---|---|
| TA 98 | TA 100 |

| | Without S9 | With S9 | Without S9 | With S9 |
|---|---|---|---|---|
| Before heating | 19 ± 1 | 34 ± 2 | 114 ± 6 | 152 ± 12 |
| After heating | 27 ± 2 | 29 ± 2 | 121 ± 6 | 126 ± 6 |

The above results demonstrated that neither the polar or non-polar fractions exhibited mutagenicity towards the His- strains, regardless of the presence of metabolic activators. Although the formation of mutagens are associated with heating and cooking conditions, rice bran oil is not detectably affected by such conditions.

Taken together, the results and discussion above show that rice bran and its product are antimutagenic, anticarcinogenic, and safe for human consumption.

All publications, patents and patent applications mentioned in this specification are herein incorporated by reference into the specification in their entirety for all purposes.

Although the invention has been described with reference to preferred embodiments and examples thereof, the scope of the present invention is not limited only to those described embodiments. As will be apparent to persons skilled in the art, modifications and adaptations to the above-described invention can be made without departing from the spirit and scope of the invention, which is defined and circumscribed by the appended claims.

TABLE 4
COMPARISON OF HEALTH BENEFITS AMONG
FIBERS AVAILABLE IN THE MARKET

| Health benefits | RiceMucil ™ | Metamucil | Citrucel | Fiber Con | Inulin |
|---|---|---|---|---|---|
| 1. Source | All natural | Natural | Synthetic | Synthetic | Natural |
| | (stabilized rice bran) | (Psyllium husk) | | | (Chicory) |
| 2. Type of fiber | Soluble & insoluble | Only Soluble | Only Soluble | Only Soluble | Soluble |
| | | fiber (>65%) | fiber | fiber | |
| 3. Health benefits | | | | | |
| Colon Health | Produces short chain | Not effective in | Not effective in | Not effective in | Not effective in |
| | fatty acid on colonic | maintaining | maintaining | maintaining | maintaining |
| | fermentation result- | colon health. | colon health. | colon health. | colon health. |
| | ing in lower pH and | Produces gases | Produces gases | Produces gases | Produces gases |
| | maintaining colon | (methane & | (methane & | (methane & | (methane & |
| | health | carbon dioxide) | carbon dioxide) | carbon dioxide) | carbon dioxide |
| | | on colonic | on colonic | on colonic | on colonic |
| | | fermentation | fermentation | fermentation | fermentation |
| | | resulting in | resulting in | resulting in | resulting in |
| | | abdominal | abdominal | abdominal | abdominal |
| | | distress | distress | distress | distress |
| Prevention of | Prevents colon cancer | Not effective | Not effective | Not effective | Not effective |
| colon cancer | | | | | |
| Improvement | Immunoregulatory effect | Not effective | Not effective | Not effective | Not effective |
| of the immune | | | | | |
| system | | | | | |
| Improvement | Helps in lowering lipid | Helps in | Helps in | Helps in | Helps in |
| of the | parameters. It traps | lowering lipid | lowering lipid | lowering lipid | lowering lipid |
| cardiovascular | the bile salts and bile | parameters | parameters | parameters | parameters |
| health | pigments and prevents | | | | |
| | its reentry into blood | | | | |
| | stream (patent) | | | | |
| Improvement | Helps in | Helps in | Helps in | Helps in | Helps in |
| of diabetes | modulating | modulating | modulating | modulating | modulating |
| | blood glucose | blood glucose | blood glucose | blood glucose | blood glucose |
| | levels (patent) | levels | levels | levels | levels |
| Improvement | Very effective | Not very | Not very | Not very | Not very |
| of Bowel | | effective | effective | effective | effective |
| function | | | | | |
| 4. Allergic | No allergies | Allergic | Not determined | Not determined | Not determined |
| reactions | Hypoallergenic product | manifestations | | | |
| 5. Gluten | Gluten free | Not determined | Not determined | Not determined | Not determined |
| 6. Antioxidants, | Potent antioxidants | Not present | Not present | Not present | Not present |
| phytonutrients, | phytonutrients, | | | | |
| vitamins and | vitamins and minerals | | | | |
| minerals | naturally present in | | | | |
| | the product | | | | |
| 7. Nutrient rich | High protein, low | No nutrient is | No nutrient is | No nutrient is | Not present |
| | carbohydrate anti- | present | present | present | |
| | oxidant fiber | | | | |

TABLE 5
RICEMUCIL

| INGREDIENTS | |
|---|---|
| A non-chemically modified water-insoluble fraction of stabilized rice | |
| bran. It contains 4 grams of insoluble fiber, several minerals, IP6, | |
| antioxidants, and 2 grams of hypoallergenic protein (per serving). | |
| GUARANTEED SPECIFICATIONS | |
| Protein | 18-22% |
| Fat | 11-16% |
| Total Carbohydrates | 50-60% |
| Total Dietary Fiber | 40-50% |
| Soluble Fiber | 0-1% |
| Ash | 8-12% |
| Moisture | 1-5% |
| Free Fatty Acids | <4% |
| MICROBIOLOGICAL | |
| Microbiologically safe and pure. | |
| PHYSICAL | |
| Appearance | light, fibrous, solid |
| Color | brown/tan |
| Flavor | bland |
| Bulk Density (g/cc) | 0.26 |
| TYPICAL ANALYTICAL DATA | |
| MACRONUTRIENTS (g/100 g) | |
| Protein | 20.50 |
| Fat | 13.50 |
| Saturated Fatty Acid | 2.40 |
| Total Carbohydrate | 52.50 |
| Available Carbohydrate | 10.50 |
| Ash | 10.00 |
| Moisture (100 degree vac.) | 3.50 |
| Crude Fiber | 12.20 |
| Total Dietary Fiber | 42.00 |
| Soluble Fiber | 0.78 |
| Calories/100 g. | 245.50 |
| VITAMINS | |
| Vitamin A; Carotenoids (mcg/100 g) | |
| α-Carotene | 2.60 |
| β-Carotene | 0.00 |
| Lycopene | 0.13 |
| Lutein | 24.80 |
| Zeaxanthin | 6.40 |
| Precryptoxanthin/Cryptoxanthin | 0.60 |
| Total Carotenoids | 34.53 |
| Vitamin B Complex (mg/100 g) | |
| Vitamin B1 | 2.00 |
| Vitamin B2 | 0.19 |
| Vitamin B3 | 30.60 |
| Vitaimn B5 | 1.90 |
| Vitamin B6 | 1.70 |
| Vitamin B12 (mcg/100 g) | <0.500 |
| Vitamin C (mg/100 g) | <0.500 |
| Vitamin E Complex (mg/100 g) | |
| Tocopherols (T) | 1.20 |
| Tocotrienols | 2.50 |
| Total Tocols | 3.70 |
| Other Micronutrients (mg/100 g) | |
| Folic Acid | <25.00 |
| Biotin | 11.04 |
| Choline | 70.50 |
| Inositol | 1314.00 |
| Gamma-Oryzanol | 174.10 |
| Phytosterols (mg/100 g) | |
| Beta-Sitosterol | 146.46 |
| Stigmasterol | 67.15 |
| Campesterol | 90.40 |
| Brassicasterol | 13.20 |
| Total Phytosterols | 317.21 |
| MINERALS (mg/100 g) | |
| Sodium | 16.00 |
| Potassium | 1670.00 |
| Calcium | 92.50 |
| Magnesium | 1223.30 |
| Phosphorous | 2330.00 |
| Manganese | 38.40 |
| Iron | 11.60 |
| Copper | 0.09 |
| Zinc | 9.34 |
| Chromium (ppm) | <1 ppm |
| Total Sugars (g/100 g) | 4.26 |
| (No Lactose) | |

1-23. (canceled)

24. A method for treating colon cancer in an individual in need thereof, said method comprising:
administering to said individual a therapeutic effective amount of a rice bran composition, to treat colon cancer.

25. The method of claim 24, wherein said composition comprises at least 35% w/w rice bran fiber.

26. The method of claim 25, wherein said composition comprises between 35% w/w rice bran fiber and 60% w/w rice bran fiber.

27. The method of claim 25, wherein said composition comprises between 35% w/w rice bran fiber and 50% w/w rice bran fiber.

28. The method of claim 24, wherein said composition is RiceMucil™.

29. The method of claim 24, wherein after administration, said composition reaches the colon, the bacteria ferment said composition, which produce a short chain fatty acid.

30. The method of claim 29, wherein said short chain fatty acid is a member selected from the group consisting of acetic, propionic and butyric acid.

31. The method of claim 29, wherein fermentation of said composition in the intestines lowers the pH.

32. The method of claim 24, wherein said composition treats said cancer at the initiation stage.

33. The method of claim 24, wherein said composition treats said cancer during a latent period by a suppressor mechanism, preventing proliferation of diseased cells.

34. The method of claim 24, wherein said composition acts on previously formed DNA adducts to suppress the progression of said cancer.

35. The method of claim 34, wherein said composition switches cell programming from a proliferative to a differentiation pathway, reversing said cancer progression.

36. The method of claim 24, wherein said composition comprises IP6.

37. The method of claim 36, wherein IP6 functions both as a blocking agent and as a suppressive agent.

38. The method of claim 24, wherein the administration of said composition leads to an increase in measured intestinal enzyme activity in said subject.

39. The method of claim 24, wherein the administration of said composition improves the absorption of sodium and water in the intestines.

40. A method for decreasing the likelihood of colon cancer in an individual, said method comprising:
administering to said individual a therapeutic effective amount of a rice bran composition, to decreasing the likelihood of colon cancer.

41. The method of claim 40, wherein said composition comprises at least 35% w/w rice bran fiber.

42. The method of claim 40, wherein said composition comprises between 35% w/w rice bran fiber and 60% w/w rice bran fiber.

43. The method of claim 40, wherein said composition is RiceMucil™.
